(19) United States
(12) Patent Application Publication
(10) Pub. No.: US 2007/0044168 A1
(43) Pub. Date: Feb. 22, 2007

(54) Transplantable tumor model

(75) Inventors: Sergei Roveskoy, Chicago, IL (US); Igor Mizgireuv, St. Petersburg (RU)

Correspondence Address: Medlen & Carroll, LLP, Suite 350, 101 Howard Street, San Francisco, CA, 94105, US

(73) Assignee: Northwestern University, Evanston, IL (US)

(21) Appl. No.: 11/485,209

(22) Filed: Jul. 12, 2006

Related U.S. Application Data
(60) Provisional application No. 60/698,608, filed on Jul. 12, 2005.

(51) Int. Cl. A01K 67/027; C12N 5/06

(52) U.S. Cl. 800/20; 435/325

(57) ABSTRACT

The present invention relates to compositions and methods for treating cancer. In particular, the present invention provides compositions and methods comprising transplantable tumor lines as a non-mammalian model of tumor growth and methods of using the same in research, diagnostic and therapeutic applications (e.g., in pharmacologic and cancer research and treatment applications).

This application claims priority to U.S. Provisional Patent Application No. 60/698,608, filed Jul. 12, 2005, hereby incorporated by reference in its entirety.

This invention was funded, in part, under NIH Grant R21-CA098856-02. The government may have certain rights in the invention.

### FIELD OF THE INVENTION

The present invention relates to systems and methods for cancer treatment and therapy. In particular, the present invention provides genetically identical fish (e.g., clonal, isogenic, congenic, and/or inbred small teleost fish) as well as systems and methods comprising transplantable tumor lines as a non-mammalian model of tumor growth, and methods of using the same in research, diagnostic and therapeutic applications (e.g., in pharmacologic and cancer research and treatment applications).

### BACKGROUND OF THE INVENTION

Cancer remains the number two cause of mortality in this country, resulting in over 500,000 deaths per year. Despite advances in detection and treatment, cancer mortality remains high. Full understanding of the etiology and causation of cancer remains elusive. This in part is attributed to a lack of models (e.g., whole animal or organism models) that have been problematic due to expense and ease of use. Thus, effective therapeutic strategies for cancers (e.g., malignant cancers) have been slow to surface.

For example, pancreatic cancer is one of the most enigmatic and aggressive malignant diseases facing oncologists (See, e.g., Parker et al., “Cancer Statistics. 1996,” CA Cancer J. Clin., 46:5-27 (1996) (“Parker”)). It is now the fourth leading cause of cancer death in both men and women in the United States, and the incidence of this disease has significantly increased over the past 20 years (See, e.g., Parker; Trede et al., Ann. Surg., 211:447-458 (1990); Cameron et al., Ann. Surg., 217:430-438 (1993); Horward, Curr. Prob. in Cancer, 20:286-293 (1996) (“Horward”); Poston et al., Gut. Biology of Pancreatic Cancer, 32:800-812 (1991) (“Poston”); and Black et al., Oncology, 10:301-307 (1996) (“Black”)). Pancreatic cancer is responsible for 27,000 deaths per year in the United States. Because of lack of early diagnosis and poor therapeutic responsiveness of pancreatic cancer, less than 2% of patients survive beyond five years, and the median expectation of life after diagnosis of pancreatic cancer is less than 6 months (See, e.g., Horward; Poston; and Black).

Colonic cancer is the second most common form of cancer in the United States (See, e.g., Doll et al., BMJ, 2:1525-1536 (1976); Hruban et al., Adv. Anat. Pathol., 5:175-178 (1998) (“Hruban”); Figueredo et al., Cancer Prev. Control, 1:379-92 (1997) (“Figueredo”); Ness et al., Am. J. Gastroenterol., 93:1491-7 (1998) (“Ness”); Trehu et al., South Med. J., 85:248-253 (1992); and Wingo et al., “Cancer Statistics,” CA Cancer J. Clin., 45:8-30 (1995) (“Wingo”)). Colonic cancer occurs in more than 138,000 patients and is responsible for more than 55,000 deaths in the United States each year (Wingo). Up to 70% of patients with colonic cancer develop hepatic metastasises by the time of death, indicating that non-detectable micro-metastases are present at the time of surgery (See, e.g., Hruban; Figueredo; and Ness). Furthermore, metastatic cancer is often not responsive to standard chemotherapeutic regimens, resulting in treatment failure (See, e.g., Figueredo and Ness). The overall response of advanced or non-resectable colorectal cancer patients to chemotherapeutic agents varies from 26 to 44 percent. For example, less than one third of colorectal cancer patients with liver metastases respond to treatment with agents such as 5-FU and leucovorin (Id.).

Breast cancer has the highest incidence of any cancer in women with the diagnosis being made in more than 275,000 per year in the USA (See, e.g., Richards et al., Lancet, 353:1119-26 (1999); Norton, Semin. Oncol., 26:1-4 (1999); Morrow et al., Curr. Probl. Surg., 36:163-216 (1999); and Ruppert et al., Breast Cancer Res. Treatment, 44:93-114 (1997)). Even though five year survival has increased to more than 80%, more than 77,000 women still die from this disease each year (Id.).

Thus, what is needed are new models of cancer (e.g., that provide the ability to identify and test new forms and targets of treatment for cancer (e.g., metastatic cancers)).

### SUMMARY OF THE INVENTION

The present invention relates to systems and methods for cancer treatment and therapy. In particular, the present invention provides genetically identical fish (e.g., clonal, isogenic, congenic, and/or inbred small teleost fish) as well as systems and methods comprising transplantable tumor lines as a non-mammalian model of tumor growth, and methods of using the same in research, diagnostic and therapeutic applications (e.g., in pharmacologic and cancer research and treatment applications).

Accordingly, in some embodiments, the present invention provides a method of identifying a test compound useful for treating cancer comprising providing a subject, wherein the subject is a zebrafish harboring transplanted tumor cells; administering a test compound to the subject; monitoring the subject; and identifying a test compound that alters a characteristic of the subject. In some embodiments, the zebrafish is selected from the group consisting of CB1 and CW1. In some embodiments, the transplanted tumor cells are selected from the group consisting of zt2, zt6, zt8, zt9, zt10, zt12, zt15, zt16, zt18, zt20, zt21, zt23, zt28, zt29, zt34, s1, s3, s10, and s11. The present invention is not limited by the type of test compound administered. Indeed, a variety of test compounds find use in the systems and methods of the present invention including, but not limited to, carbohydrates, monosaccharides, oligosaccharides, polysaccharides, amino acids, peptides, oligopeptides, polypeptides, proteins, nucleosides, nucleotides, oligonucleotides, polynucleotides (e.g., DNA and DNA fragments, RNA and RNA fragments and the like), lipids, retinoids, steroids, drugs, antibodies, prodrugs, glycopeptides, glycoproteins, proteoglycans and the like, and synthetic analogues or derivatives thereof, including peptidomimetics, small molecule organic compounds and the like, and mixtures and/or combinations thereof, as well as others described herein. In some embodiments, the characteristic monitored is transplanted tumor cell growth. In some embodiments, the characteristic monitored is transplanted tumor cell mass. In some embodiments, the characteristic monitored is zebrafish mass. In some embodiments, the characteristic monitored is zebrafish mortality. In some embodiments, the characteristic monitored is expression of a cancer biomarker. The present invention is not limited to any particular biomarker. Indeed, a variety of cancer biomarkers are described herein. In some embodiments, the expression monitored is gene expression. In some embodiments, the expression monitored is protein expression. In some embodiments, protein activity is monitored. In some embodiments, the characteristic monitored is the number of transplanted tumor cells. In some embodiments, microscopy is used for monitoring. The present invention is not limited to any particular type of microscopy. Indeed, a variety of microscopic techniques may be used including, but not limited to, direct microscopy, dissecting microscopy, fluorescent microscopy, and inverted microscopy. Similarly, the type of zebrafish monitored is not limiting. For example, in some embodiments, zebrafish monitored are zebrafish embryos. In some embodiments, zebrafish monitored are zebrafish larvae. In some embodiments, zebrafish monitored are adult zebrafish. The present invention can be used in any one of a number of screening formats. For example, compositions and methods of the present invention can be used in a high through-put screen to identify test compounds with anticancer and/or anti-tumor properties. In some embodiments, zebrafish are present within a multi-well plate. In some embodiments, monitoring comprises histological techniques. The present invention is not limited to the type of histological technique utilized. For example, histological techniques useful in the present invention include, but are not limited to fixation of the material, decalcination and paraffination, and preparation and staining tissue sections.

The present invention also provides a method of identifying a test compound with anti-cancer and/or antitumor properties comprising administering to an isogenic zebrafish harboring transplanted tumor cells a test compound and monitoring the test compound's ability to alter transplanted tumor cell presence and/or growth in the subject.

The present invention also provides one or more tumor cell lines. In some embodiments, the tumor cell lines are derived from a homozygous zebrafish clone. For example, clones from which the tumor cell lines are derived include, but are not limited to, tumor cell lines zt2, zt6, zt8, zt9, zt10, zt12, zt15, zt16, zt18, zt20, zt21, zt23, zt28, zt29, zt34, s1, s3, s10, and s11. In some embodiments, the tumor cell line retains the ability to form tumors in a subject after a cycle of freezing and thawing. In some embodiments, the tumor cell line is derived from a zebrafish clone selected from the group consisting of CB1 and CW1. In some embodiments, the present invention provides methods of inducing a tumor in clonal, isogenic, congenic, and/or inbred small teleost fish. The present invention is not limited to any particular method of inducing a tumor. Indeed, a variety of methods may be used including, but not limited to, chemical carcinogens (e.g., DEN described below), physical means (e.g., irradiation, UV-light, etc.), via introduction of nucleic acid constructs expressing tumorigenic agents (e.g., oncogenes), and other methods known in the art.

The present invention also provides a method of generating genetically identical (e.g., clonal, isogenic, congenic, and/or inbred) small teleost fish (e.g., zebrafish) comprising: a) providing eggs from female strains of small teleost fish (e.g., AB or brass strains of zebrafish fertilized by UV-inactivated sperm from male zebrafish), b) heat-shocking the eggs, wherein the heat-shocking blocks the first cleavage of the fertilized eggs; c) obtaining eggs from female homozygous diploid fish resulting from steps (a) and (b); d) repeating steps (a) and (b) with the eggs of step (c) to generate clones of homozygous fish; and e) crossing two different homozygous clones of the same maternal origin, thereby generating an isogenic zebrafish. Thus, in some embodiments, the present invention also provides clonal, isogeneic, congenic, inbred or close to inbred lines of small teleost fish (e.g., zebrafish and medaka). In some embodiments, the present invention provides a genetically identical strain (e.g., clonal, isogenic, congenic, and/or inbred strain) of small teleost fish (e.g., zebrafish) generated according to methods described herein. The present invention is not limited by the genetically identical small telost fish generated. Indeed, a variety of small teleost fish can be generated utilizing the methods described herein including, but not limited to, zebrafish (e.g., strains CB1 and CW1). In some embodiments, the present invention provides genetically identical fish (e.g., clonal, isogeneic, congenic, inbred or close to inbred lines (e.g., lines generated via methods utilized to inbreed rodents (e.g., mice) of small teleost fish (e.g., zebrafish and medaka))). In some embodiments, the present invention provides for the induction of tumors in genetically identical fish. In some embodiments, the present invention provides the generation of transplantable tumor lines derived from tumors induced in the genetically identical fish. In some embodiments, the present invention provides a method of characterizing a test compound (e.g., for anticancer and/or antitumor properties) in a subject, wherein the subject is a clonal, isogeneic, congenic, inbred or close to inbred line of a small teleost fish (e.g., zebrafish and medaka).

### DESCRIPTION OF THE DRAWINGS

FIG. 1 shows the growth of transplantable and spontaneous tumors in juvenile and adult zebrafish. Advanced stages of tumor development after intraperitoneal (i.p.) (A) and intramuscular (i.m.) (B) transplantation of tumors zt6 and zt34, respectively. Typical enlargement of frontal abdominal wall as a result of tumor growth (arrowhead). C, tumor mass (arrowhead) penetration of abdominal wall after i.p. transplantation of tumor zt18. D, infiltrating growth of s1 tumor after i.p. transplantation. Amorphous tumor mass fills in the entire abdomen invading inner organs. E, tumor zt15 after i.p. transplantation. The tumor is growing as an isolated nodule pressing on surrounding tissues. F, formation of liver-like structure in result of growth of “minimal” hepatoma zt20 after i.p. transplantation. Age of transplant is 8 weeks. G, primary spontaneous acinar cell carcinoma of pancreas s10 developed in 11-month-old female zebrafish of CB1 line (T, tumor nodules). H, tumor zt34 developed after i.p. transplantation into isogeneic host fish. I and J, 23-day-old juvenile fish through 11 days after i.p. injection of either PBS (I) or zt23 tumor cell suspension (J). The tumor-bearing fish reveals enlarged, compare with control fish, peritoneal cavity filled with opaque tumor mass (arrowhead) penetrating abdominal wall (arrow). K, sagittal histological section of 23-day-old juvenile fish through 11 day after i.p. injection of zt23 tumor cell suspension. Tumor mass (T) fills in almost the entire peritoneal cavity, infiltrating abdominal organs (L, liver; SB, swim bladder). L, zt23 cell suspension and cell clusters just before the transplantation (white field microscopy).

FIG. 2 shows the histological structure of transplanted lines of tumors in zebrafish.

FIG. 3 shows the lifespan of tumor-bearing fish. A, i.p. transplantation of tumor zt23 (▪) and s1 (▴) to 2.5-month-old fish. B, i.p. zt23 tumor transplantation (▪) to 12-day larvae. Control intact fish (●)

Definitions

To facilitate an understanding of the present invention, a number of terms and phrases are defined below.

The terms “test compound” and “candidate compound” refer to any chemical entity, pharmaceutical, drug, and the like that is a candidate for use to treat or prevent a disease, illness, sickness, or disorder of bodily function (e.g., cancer (e.g., tumors)). Test compounds comprise both known and potential therapeutic compounds. A test compound can be determined to be therapeutic by screening using the screening methods of the present invention. Examples of test compounds include, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, drug, antibody, prodrug, glycopeptides, glycoproteins, proteoglycans and the like, and synthetic analogues or derivatives thereof, including peptidomimetics, small molecule organic compounds and the like, and mixtures thereof (e.g., that is a candidate for use to treat or prevent a disease, illness, sickness, or disorder of bodily function (e.g., cancer (e.g., tumors)). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention. In some embodiments, “test compounds” are anticancer and/or antitumor agents. In some embodiments, “test compounds” are anticancer agents that induce apoptosis in cells.

As used herein, the term “heterologous” refers to any cell or tissue from a different organism, or from the same organism that is not in its natural environment. For example, a heterologous cell or tissue (e.g., cancerous cell or tumor) includes a cell or tissue from one species (e.g., strain) introduced into another species. A heterologous cell or tissue also includes a cell or tissue native to an organism that has been altered in some way.

As used herein, the term “test compound library” refers to a mixture or collection of one or more compounds generated or obtained in any manner. Preferably, the library contains more than one compound or member. The test compound libraries employed in this invention may be prepared or obtained by any means including, but not limited to, combinatorial chemistry techniques, fermentation methods, plant and cellular extraction procedures and the like. Methods for making combinatorial libraries are well-known in the art (See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Each of these references is incorporated herein by reference in its entirety).

The term “synthetic small molecule organic compounds” refers to organic compounds generally having a molecular weight less than about 1000, preferably less than about 500, which are prepared by synthetic organic techniques, such as by combinatorial chemistry techniques.

As used herein the term “prodrug” refers to a pharmacologically inactive derivative of a parent “drug” molecule that requires biotransformation (e.g., either spontaneous or enzymatic) within the target physiological system to release, or to convert (e.g., enzymatically, mechanically, electromagnetically, etc.) the “prodrug” into the active “drug.” “Prodrugs” are designed to overcome problems associated with stability, toxicity, lack of specificity, or limited bioavailability. Exemplary “prodrugs” comprise an active “drug” molecule itself and a chemical masking group (e.g., a group that reversibly suppresses the activity of the “drug”). Some preferred “prodrugs” are variations or derivatives of compounds that have groups cleavable under metabolic conditions. Exemplary “prodrugs” become pharmaceutically active in vivo or in vitro when they undergo solvolysis under physiological conditions or undergo enzymatic degradation or other biochemical transformation (e.g., phosphorylation, hydrogenation, dehydrogenation, glycosylation, etc.). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common “prodrugs” include acid derivatives such as esters prepared by reaction of parent acids with a suitable alcohol (e.g., a lower alkanol), amides prepared by reaction of the parent acid compound with an amine (e.g., as described above), or basic groups reacted to form an acylated base derivative (e.g., a lower alkylamide).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

As used herein, the term “sample” is used in its broadest sense. In one sense, it is meant to include a specimen or culture obtained from any source, as well as biological and environmental samples. Biological samples may be obtained from fish and/or animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather than a particular structure such as an epitope).

As used herein, the term “subject” refers to any fish and/or animal (e.g., a mammal), including, but not limited to, humans, non-human primates, rodents, and the like.

As used herein, the term “characterizing cancer in subject” refers to the identification of one or more properties of a cancer sample in a subject, including but not limited to, the presence of benign, pre-cancerous or cancerous tissue and the stage of the cancer. Cancers may be characterized by characterizing transplantable cell lines of the present invention.

As used herein, the term “characterizing tissue in a subject” refers to the identification of one or more properties of a tissue sample.

As used herein, the term “effective amount” refers to the amount of a composition (e.g., a test compound) sufficient to effect beneficial or desired results (e.g., prolonged life of a subject and/or decreased tumor burden in a subject). An effective amount can be administered in one or more administrations, applications or dosages and is not intended to be limited to a particular formulation or administration route.

As used herein, the term “administration” refers to the act of giving a drug, prodrug, or other agent (e.g., a test compound), or therapeutic treatment (e.g., compositions of the present invention) to a subject (e.g., a subject or in vivo, in vitro, or ex vivo cells, tissues, and organs). Exemplary routes of administration may be through the eyes (ophthalmic), skin (transdermal), by injection (e.g., intramuscularly, intravenously, subcutaneously, intratumorally, intraperitoneally, etc.) and the like.

As used herein, the term “co-administration” refers to the administration of at least two agent(s) (e.g., a test compound and one or more other agents) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., a test compound) with a carrier, inert or active, making the composition especially suitable for therapeutic use in vitro, in vivo or ex vivo.

The terms “pharmaceutically acceptable” or “pharmacologically acceptable,” as used herein, refer to compositions that does not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference in its entirety).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

The term “isolated” when used in reference to a cell refers to a cell that is removed from its natural environment (e.g., a solid tumor) and that is separated (e.g., is at least about 75% free, and most preferably about 90% free), from other cells with which it is naturally present, but that lack the marker based on which the cells were isolated.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “cell culture” refers to any in vitro culture of cells. Included within this term are continuous cell lines (e.g., with an immortal phenotype (e.g., isogenic cancer cell lines described herein)), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

### DETAILED DESCRIPTION OF THE INVENTION

The present invention relates to systems and methods for cancer treatment and therapy. In particular, the present invention provides genetically identical fish (e.g., clonal, isogenic, congenic, and/or inbred small teleost fish) as well as systems and methods comprising transplantable tumor lines as a non-mammalian model of tumor growth and methods of using the same in research, diagnostic and therapeutic applications (e.g., in pharmacologic and cancer research and treatment applications).

The present invention provides compositions comprising tumor lines (e.g., transplantable cell lines) derived from engineered tumors (e.g., spontaneous, induced or genetically engineered). The present invention further provides fish lines (e.g., clonal or non-clonal but genetically identical (e.g., isogenic)) comprising the tumor lines (e.g., Danio rario (i.e., zebrafish) or Oryzias latipes (i.e., medaka)). Thus, the present invention provides novel, non-mammalian models (e.g., clonal, isogenic, congenic, and/or inbred small teleost fish (e.g., for the characterization and identification of treatments for cancer and/or tumors)).

Transplantable tumor lines remain one of the main models for investigation of different aspects of tumor biology in rodents (See, e.g., Klein G. Cancer Res 1959;19:343-58). Progress in the generation of sustainable transplantable tumors in fish (e.g., zebrafish) has been limited because of the lack of a true inbred zebrafish strain as a result of loss of fertility during inbreeding (See, e.g., Trede et al., Immunity 2004;20: 367-79). In order to overcome this obstacle, two separate clonal homozygous lines of zebrafish were generated in which the viability and fertility of each was comparable with that of wild-type fish (See, e.g., Example 1). Although an understanding of the mechanism is not necessary to practice the present invention and the present invention is not limited to any particular mechanism of action, the lack of genetic differences between individual fish within a clone enables successful passaging (e.g., multiple passages) of tumors (e.g., DEN-induced and spontaneous tumors) in zebrafish. Accordingly, in some embodiments, the present invention provides genetically identical fish (e.g., isogenic zebrafish (e.g., in which the viability and fertility of the fish is similar to that of wild-type fish (See, e.g., Example 1))). However, the present invention is not limited to the generation of isogenic fish. The present invention also provides the generation of clonal, congenic, and/or inbred small teleost fish (e.g., zebrafish and Medaka). Various examples are provide below with regard to methods of using isogenic fish (e.g., for characterizing test compounds). However, it should be appreciated that other types of genetically identical fish (e.g., clonal, congenic, and/or inbred small teleost fish) may also be used in similar methods for characterizing test compounds and/or for generation of tumors and tumor cell lines. For example, in some embodiments, it may be desired to generate fish that are genetically identical at certain loci (e.g., a histocompatibility complex gene or other molecule involved in immune system function) but that are not genetically identical at other loci in their genomes. Such fish also find use in the present invention. For example, methods of the present invention may be utilized to generate fish that are not isogenic, but nonetheless are genetically identical at one or more loci in their genome (e.g., that can be utilized for serial transplantation (e.g., of cells, tissues, organs, etc.) or for experimentation).

The majority of DEN-induced tumors in clonal zebrafish were liver-derived tumors. One exception was a carcinoma of pancreas. This finding was surprising because prior to experiments conducted during the development of the present invention, benign pancreatic tumors were described in zebrafish treated with N-methyl-N′-nitro-N-nitrosoguanidine (See, e.g., Spitsbergen et al., Toxicol Pathol 2000;28:705-15) and 7,12-dimethylbenz(a)anthracene (See, e.g., Spitsbergen et al., Toxicol Pathol 2000; 28:716-25) but not with DEN (See, e.g., Khudoley, J Natl Cancer Inst Monogr 1980; 65:65-70). Four spontaneous tumors diagnosed as acinar cell carcinomas of pancreas were detected in 10.5- to 18-month-old female CB1 zebrafish. Considering that spontaneous tumors of pancreas occur very seldom in zebrafish (See, e.g., Smolowitz et al., Biol Bull 2002; 203:265-6), CB1 fish might have genetic predisposition to development of pancreatic tumors (e.g., because of a possible loss of heterozygosity of tumor suppressor genes; See, e.g., Nikiforova et al., Clin Endocrinol 1999; 51:27-33) as a result of homozygous nature of their genome.

The majority of DEN-induced and spontaneous tumors that emerged in CB1 zebrafish showed growth after grafting to both syngeneic and isogeneic but not to wild-type animals. Thus, although an understanding of the mechanism is not necessary to practice the present invention and the present invention is not limited to any particular mechanism of action, these results indicate that, in some embodiments, immunogenicity of transplantable tumors in zebrafish is determined by their histocompatibility complex (See, e.g., Ono et al., Proc Natl Acad Sci U S A 1992; 89:11886-90) and therefore is highly similar to that of mammalian tumors (See, e.g., Snell, Cancer Res 1952; 12:543-6). Currently, of 19 transplantable cancers (e.g., tumor cell lines) that were generated during the development of the present invention, 12 lines have undergone from 10 to 25 passages. These tumors retain their morphology and growth rate that are hallmarks of stable tumor lines. The tumor lines had significant differences in their growth rate and in other biological features just as occurs in the rodent model (See, e.g., Reuber, GANN Monogr 1961; 6:43-54). Some transplantable tumors generated during development of the present invention underwent growth arrest after several consecutive passages. In such cases, necropsy did not reveal visible tumor connection to the recipient's vascular system. Thus, although an understanding of the mechanism is not necessary to practice the present invention and the present invention is not limited to any particular mechanism of action, in some embodiments, such tumors lack an ability to promote angiogenesis playing a vital role in carcinogenesis (See, e.g., Carmeliet and Jain, Nature 2000; 407:249-57). The growth rate of tumors derived from spontaneous (e.g., line s1) and DEN-induced (e.g., line zt23) carcinomas in pancreas was similar to that of the most fast growing tumor lines in mammalians (See, e.g., Dawe and Potter, Am J Pathol 1957; 33:603).

Accordingly, in some embodiments, composition and methods of the present invention can be used for the quantitative assessment of cancer cell (e.g., tumor) development (e.g., in a transplant model). For example, in some embodiments, the present invention provides tumor cell lines (e.g., transplantable tumor cell lines) and genetically identical (e.g., clonal, isogenic, congenic, and/or inbred) small teleost fish for use in systems and methods of cancer research and treatment (e.g., that can be used to identify and characterize test compounds for use in cancer treatment (e.g., as cancer chemotherapies and/or anti-tumor drugs)).

In some embodiments, a mean survival time of tumor-bearing adult fish is used for screening test compounds and/or agents (e.g., to identify and/or characterize a test compound). In some embodiments, a mean survival time of tumor-bearing 5- to 14-day-old larvae grafted with tumor cells is used (e.g., to identify and/or characterize a test compound (e.g., as a criterion of treatment efficacy)). However, the present invention is not so limited. For example, further refinement of this technology could be achieved by, for example, injecting tumor cells constitutively expressing one or more fluorescent tags (See, e.g., Langenau et al., Science 2003; 299:887 90; Lyons, J Pathol 2005; 205:194-205) into embryos and early larvae. The small size and transparent body of the developing zebrafish make this approach applicable to high-throughput screening assays (e.g., for use in multiwell (e.g., a 96- or 24-microwell) format (See, e.g., Love et al., Curr Opin Biotechnol 2004; 15:564-71). Moreover, in some embodiments, methods used in rodents to assess tumor growth can be readily adapted to small teleost fish (e.g., zebrafish). For example, experiments conducted during the development of the present invention provide that the dynamics of body weight gain in tumor-bearing fish can also be used for quantitative assessment of tumor growth in small teleost fish (e.g., zebrafish). Thus, in some embodiments, a direct measurement of the size of i.m. transplanted tumors can be utilized (e.g., using computer morphometry).

The present invention also provides that tumor lines generated and characterized herein can be stored (e.g., frozen) and utilized at a later time. For example, the successful transplantation of zt23, zt34, and s1 tumors recovered after cryopreservation in liquid nitrogen showed the possibility of long-term storage of zebrafish tumor lines. Thus, in some embodiments, it is unnecessary to maintain tumor lines by routine serial transplantation in live fish (e.g., thereby reducing labor and cost of maintenance of large number of tumor lines).

Thus, the present invention provides the identification and characterization of transplantable fish tumor lines (e.g., tumor lines in zebrafish) that display high similarities to characteristics of rodent tumors. For example, no specific biological features of zebrafish tumors were identified that were altered in a significant way from mammalian tumors (See, e.g., Klein G. Cancer Res 1959; 19:343-58). The present invention provides characterization of the similarity of the molecular, cellular, and immune mechanisms of tumor formation in fish and mammals. Thus, the compositions and methods of the present invention find use in research and therapeutic settings (e.g., that until the present invention used mammalian models as the experimental tool of choice). In some embodiments, the present invention provides genetically identical subjects that opens new opportunities for investigations and research in tumor immunology (See, e.g., Ostrand-Rosenberg Curr Opin Immunol 2004; 16:143-50) and cancer genetics (See, e.g., Streisinger J Natl Cancer Inst Monogr 1984; 65:53-8). In some embodiments, the present invention provides compositions and methods suitable for investigation of host-tumor interactions.

In some embodiments, the present invention provides compositions (e.g., genetically identical (e.g., clonal, isogenic, congenic, and/or inbred) fish lines) for studying of mechanisms of carcinogenesis. In some embodiments, the genetically identical fish lines are used for high throughput screening of test compounds (e.g., anticancer drugs and/or antitumor therapies). Unlike transplanted tumors in mammalians (e.g., cells or animals), in some embodiments, the present invention provides the utilization of fish embryos and larvae (e.g., as opposed to adult animals) for modeling (e.g., grafting) and analyzing transplantable tumors.

The present invention provides that zebrafish as well as medaka embryos and larvae are able to develop in vitro, have small size and a transparent body making it possible to monitor the development and spreading of cancer (e.g., tumors) in the recipient animals by relatively simple non-invasive methods (e.g., visual examination under light microscope and/or under fluorescent microscope). For example, in some embodiments, the present invention provides tumor cell lines utilizing GFP/YFP/RFP or other fluorescent reporters (e.g., luciferase). Furthermore, computer morphometry can be used for automatic quantitative assessment of tumor development (e.g., size and/or weight) and efficacy of anticancer and/or antitumor therapy (e.g., before, during and/or after administration of a test compound described herein). In some embodiments, the average time of cancer (e.g., tumor) development in embryos and larvae does not exceed 7-15 days (e.g., depending upon age at the time of transplantation). The latter feature in conjunction with a possibility of obtaining a large quantity of fertilized eggs and high throughput microinjection technique (e.g., up to 500 individual animals per hour) make the model of the present invention suitable for large scale screenings of test compounds (e.g., candidate therapeutic compounds or molecules (e.g., anti-cancer compounds, small molecules or low molecular weight compounds)).

In some embodiments, the small size of the embryos and larvae allows their maintenance throughout the entire experiment in 96-, 24- or 6-well plates, Petri dishes (e.g., 6 and 10 cm plates), or 100-150 ml beakers (e.g., that hold up to 50-150 ml medium with dissolved test compounds in it). In some embodiments, the systems of the present invention (e.g., being a direct model of cancer (e.g., tumor) growth in vertebrates) allow a simultaneous assessment of anticancer efficacy, toxicity and side effects of test/candidate compounds/molecules. In some embodiments, the anticancer efficacy, toxicity and side effects of test/candidate compounds/molecules are compared with both labor consumption and the compound quantity.

It is contemplated that the compositions and systems of the present invention provide a superior model to any existing models due to the lack of any genetic (e.g., immunogenetic) difference between tumors and tumor recipient animals. Thus, systems of the present invention provide promising compositions and methods for investigation of different approaches towards biotherapy of cancer (e.g., tumors) that are not available in mammalian models because of a frequent incomplete immune tolerance of inbred rodent strains towards available transplantable tumor lines. Thus, in some embodiments, the systems and methods of the present invention find use with transplanted cancer (e.g., tumors) (e.g., those presently transplanted in mice or rats) with much higher efficacy as a result of low cost and less labor required for fish maintenance. In some embodiments, the efficacy can be increased even further by using automatic microinjectors for tumor grafting and by application of computer morphometry and microfluorimetry for tumor growth assessment. Thus, in some embodiments, compositions and methods of the present invention can be used with a completely automated system (e.g., to identify and/or characterize anticancer and/or antitumor compounds and/or agents).

In some embodiments, the present invention provides tumor-prone small teleost fish (e.g., zebrafish) lines carrying ribosomal protein gene mutations generated as a result of mutagenesis (e.g., directed mutagenesis). In some embodiments, the present invention provides fish lines (e.g., comprising transplanted tumor lines) as a supplement for already established models of tumor development in small teleost fish (e.g., zebrafish) and as a source of transformed cell lines maintained ex vivo. Thus, the present invention provides the ability to transplant tumors from one fish to another in vivo in inbred strains.

In comparison with currently known techniques for transplantation of tumors, the present invention provides many advantages. For example, unlike methods utilizing live-bearing fish Poecilliopsis, the present invention provides genetically identical lines of egg-laying fish whose entire developmental cycle can occur in vitro. Such biological feature of these species makes their embryos and larvae, in addition to adult animals, suitable for tumor transplantation. Furthermore, females of fish species (e.g., zebrafish) are capable of laying thousands of eggs weekly, a number sufficient for any practical needs and any study design. This provides an opportunity to dramatically increase throughput capacity of tests including high throughput screening of chemical libraries and other potential treatments. In addition, a successful grafting of tumors to genetically identical fish does not require recipient's sublethal gamma-irradiation that limits a test setting to standard conditions of maintenance (e.g., fish tanks where irradiated tumor-bearing fish cannot be used in chemotherapy experiments/testing because of their low viability and even death caused by non-toxic factors for normal fish and by frequent recurrent infections). Furthermore, irradiation introduces yet another variable in analysis of relevant data and significantly increases cost—labor of data collection in addition to a requirement of access to the special equipment. In contrast to transplantation of xenogeneic (e.g. human) tumors/tumor cells into early embryos that lack immune surveillance, an approach of the present invention is not limited by this relatively short stage of animal development and gives an opportunity of long-term maintenance and observation of the grafted tumor. Therefore, systems of the present invention provide more flexibility regarding the age and developmental stage of recipient fish to which the syngeneic tumor is transplanted. In particular, according to observations made during development of the present invention, a transplantation of syngeneic tumors to 10-day larvae, as opposed to early embryos, results in much higher survival reaching 100% in recipient fish with no developmental anomalies. By contrast, a transplantation of syngeneic as well as xenogeneic tumors to embryos at the 1,000-cell stage results in mass death of recipients and multiple developmental anomalies that complicate interpretation of data. Furthermore, although in principle the growth of tumors transplanted onto embryos is possible (See, e.g., Phylonix Inc., U.S. Pat. No. 6,761,876, hereby incorporated by reference in its entirety) the host environment of mammalian cells in the latter case is far from being optimal considering different temperature, osmotic pressure and other parameters in fish. In syngeneic tumor-recipient microenvironment the modifying effect of host factors on tumor growth is minimal.

Thus, the present invention provides significant advantages over presently available technologies that are utilized for tumor transplantation in cancer investigations.

During development of the present invention, clonal homozygous lines of zebrafish were obtained and utilized (See, e.g., Example 1). Each line comprised fish that were exact genetic copies of each other, making it possible to carry out transplantation of any tissue between individual fish, without transplant rejection. Thus, in some embodiments, the present invention provides data demonstrating efficient generation of tumor cell lines (e.g., transplantable tumors) in zebrafish (See, e.g., Table 1).

The present invention provides that, in some embodiments, the qualitative assessment of tumor growth can be monitored by visual examination of tumor-bearing fish (adult animals), and by either dissecting or inverted microscopes (embryos and fries). In adult animals, in some embodiments, general signs of tumor growth are grossly visible signs of advanced stages of tumor growth (e.g., significant enlargement—often asymmetric—of the abdomen, development of ascit in the abdominal cavity, tumor penetration of the abdominal wall, cachexia or various combinations of these symptoms). In some embodiments, the size of the tumor is essentially the main criterion of assessment of tumor growth after transplantation. For example, in fries, tumor transplantation typically causes enlargement of the abdomen, filling the abdominal cavity with opaque tumor mass, penetration of the abdominal wall and further growth outside the abdominal cavity, spinal malformation (round back posture) and swimming in a non-coordinated manner. When the tumor cells utilized for transplantation express fluorescent proteins (e.g. GFP, RFP and others) or are stained with infra-vital fluorescent dyes (e.g. DiI) the tumor growth and spreading can be studied with either direct or inverted fluorescent microscope and with dissecting microscope equipped with a fluorescent module. In embryos, progression of the transplanted cancer (e.g., tumor) can be detected as the opaque mass of tumor cells gradually enlarging at the injection site (e.g. in yolk sack) or distant from it when tumor cell dissemination occurs. In addition, the developing tumor can cause embryonic malformation.

Thus, in some embodiments, a quantitative assessment of tumor growth after transplantation can be carried out by standard criteria and procedures developed for mammals. In particular, survival time of tumor-bearing animals, an increment of the body weight gain compared to control or direct measurement of tumor size by using computer-assisted morphometry after tumor grafting can be used to monitor tumor progression in living fish.

Fluorescent microscopes equipped with a high resolution digital or analogue cameras and special software designed for morphometric studies (e.g. ImagePro 4, Media Cybernetics, USA) can be used in fries and juvenile fish up to 1.5-month age for quantitative assessment of tumor progression of transplanted tumors genetically engineered to express fluorescent proteins. Tumor development can also be evaluated by standard histological technique including fixation of the material, decalcination and paraffination of the whole fish (as well as tumor nodules and complexes of tumors with surrounding tissues), preparation and staining tissue sections. Agarose blocks containing either embryos or fries (e.g., up to 3-weeks of age) can be mounted on a single slide.

In some embodiments, fish maintenance after tumor transplantation depends upon age and size of recipient fish and the design of the overall experiments. For example, in some embodiments, fish are placed in constant-water-flow systems comprising small (1-5 L) plastic or glass tanks, in 100-250 ml beakers, in plastic/glass Petri dishes or in 96-well (3-5 embryos/one 5-10-day fry per well), 24 (3-4 fries of 5-14-day age per well) or 6-well (10-12 fries of 5-14-day age per well) plates. Desired chemical compounds for testing (e.g., test compounds) can be added directly to the water as a constant delivery and/or as single or multiple pulses. Alternatively, test compounds can be injected (e.g., i.p. or i.m.) to adult fish and fries. Concentrations or doses of the compounds can be determined from preliminary toxicological studies carried out in adult fish or fries or using one or more concentrations (e.g., those suggested by the manufacture) of a specific compound or a chemical library.

In some embodiments, the present invention provides a method of identifying a test compound useful for treating cancer comprising providing a subject (e.g., genetically identical (e.g., clonal, isogenic, congenic, and/or inbred small teleost fish (e.g., zebrafish)) harboring transplanted cancer (e.g., tumors); administering a test compound to the subject; monitoring the subject (e.g., determining the weight of the subject (e.g., using morphometry), monitoring growth of the tumor (e.g., using microscopy) and/or monitoring survival of the subjects); and identifying a test compound that alters a characteristic of the cancer/tumor in the subject. The present invention is not limited to any particular characteristic altered. For example, in some embodiments, administration of a test compound inhibits growth of the tumor and/or cancer (e.g., cancer cells) in the subject (e.g., compared to a subject not receiving the test compound). In some embodiments, administration of a test compound inhibits gain of mass related to cancer and/or tumor in the subject (e.g., compared to a subject not receiving the test compound). In some embodiments, administration of a test compound increases the lifespan of the subject (e.g., compared to a subject not receiving the test compound). In some embodiments, administration of a test compound alters gene expression associated with cancer and/or tumor development. In some embodiments, gene expression is monitored. In some embodiments, monitoring gene expression identifies a cancer stem cell biomarker. In some embodiments, monitoring gene expression comprises use of a microarray. In some embodiments, monitoring gene expression comprises measuring mRNA.

In some embodiments, the response of the transplanted tumor (e.g., to the administered test compound) is monitored. In some embodiments, monitoring the response of the transplanted tumor comprises monitoring protein expression and/or activity in the tumor. In some embodiments, monitoring protein expression and/or activity in the tumor comprises use of an antibody. In some embodiments, monitoring the response of the tumor comprises monitoring cellular pathways. In some embodiments, monitoring cellular pathways comprises measuring the activity of the pathways. In some embodiments, the growth of the tumor is monitored in vitro. In some embodiments, the growth of the tumor is monitored in vivo in a subject. The present invention is not limited by the type of subject or sample (e.g., transplanted tumor or metastasis thereof) monitored.

The present invention is not limited by the type of test compound. In some embodiments, the test compound is one of a library of test compounds. The present invention is not limited by the type of test compound assayed. Indeed a variety of test compounds can be analyzed by the present invention including, but not limited to, any chemical entity, pharmaceutical, drug, known and potential therapeutic compounds, small molecule inhibitors, pharmaceuticals, a test compound from a combinatorial library (e.g., a biological library; peptoid library, spatially addressable parallel solid phase or solution phase library; synthetic library (e.g., using deconvolution or affinity chromatography selection), and the like. Examples of test compounds useful in the present invention include, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like, and synthetic analogues or derivatives thereof, including peptidomimetics, small molecule organic compounds and the like, and mixtures thereof.

The present invention also provides a method of identifying a test compound with anti-cancer and/or antitumor properties comprising administering to a subject (e.g., isogenic zebrafish) harboring transplanted cancer cells (e.g., tumor cells) a test compound and monitoring the test compound's ability to alter transplanted cancer cell (e.g., tumor cell) presence and/or growth in the subject.

Thus, in some embodiments, the present invention provides drug screening assays (e.g., to screen for anticancer and/or antitumor agents). In some embodiments, the screening methods of the present invention utilize the monitoring of biomarkers (e.g., genes and/or proteins whose expression has been associated with cancer). The present invention is not limited by the biomarker monitored. Indeed, any one of a number of biomarkers can be monitored (e.g., before, during and/or after administration of a test compound to a subject (e.g., harboring a transplanted tumor) of the present invention, including, but not limited to, HER 2 (P185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Other cancer biomarkers are described in U.S. Pat. No. 5,776,427, hereby incorporated by reference in its entirety.

Cancer biomarkers can be classified in a variety of ways. Cancer biomarkers include antigens encoded by genes that have undergone chromosomal alteration. Many of these biomarkers are found in lymphoma and leukemia. Even within this classification, biomarkers can be characterized as those that involve activation of quiescent genes. These include BCL-1 and IgH (Mantel cell lymphoma), BCL-2 and IgH (Follicular lymphoma), BCL-6 (Diffuse large B-cell lymphoma), TAL-1 and TCR-delta or SIL (T-cell acute lymphoblastic leukemia), c-MYC and IgH or IgL (Burkitt lymphoma), MUN/IRF4 and IgH (Myeloma), PAX-5 (BSAP) (Immunocytoma).

Other cancer biomarkers that involve chromosomal alteration and thereby create a novel fusion gene and/or protein include RARoa, PML, PLZF, NPMor NuM4 (Acute promyelocytic leukemia), BCR and ABL (Chronic myeloid/acute lymphoblastic leukemia), MLL (HRX) (Acute leukemia), E2A and PBXor HLF (B-cell acute lymphoblastic leukemia), NPM, ALK (Anaplastic large cell leukemia), and NPM, MLF-1 (Myelodysplastic syndrome/acute myeloid leukemia).

Other cancer biomarkers are specific to a tissue or cell lineage. These include cell surface proteins such as CD20, CD22 (Non-Hodgkin's lymphoma, B-cell lymphoma, Chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (Epithelial and lymphoid malignancies), Human leukocyte antigen (HLA)-DR, HLA-DP, and HLA-DQ (Lymphoid malignancies), RCAS1 (Gynecological carcinomas, bilary adenocarcinomas and ductal adenocarcinomas of the pancreas), and Prostate specific membrane antigen.

Tissue- or lineage-specific cancer biomarkers include epidermal growth factor receptors (high expression) such as EGFR (HER1 or erbB1) and EGFRvIII, erbB2 (HER2 or HER2/neu), erbB3 (HER3), and erbB4 (HER4).

Tissue- or lineage-specific cancer biomarkers also include cell-associated proteins such as Tyrosinase, Melan-A/MART-1, tyrosinase related protein (TRP)-1/gp75, Polymorphic epithelial mucin (PEM), and Human epithelial mucin (MUC1).

Tissue- or lineage-specific cancer biomarkers also include secreted proteins such as Monoclonal immunoglobulin, Immunoglobulin light chains, alpha-fetoprotein, Kallikreins 6 and 10, Gastrin-releasing peptide/bombesin, and Prostate specific antigen.

Still other cancer biomarkers are cancer testis (CT) antigens that are expressed in some normal tissues such as testis and in some cases placenta. Their expression is common in tumors of diverse lineages and as a group the biomarkers form targets for immunotherapy. Examples of tumor expression of CT antigens include, but are not limited to, MAGE-A1, -A3, -A6, -A12, BAGE, GAGE, HAGE, LAGE-1, NY-ESO-1, RAGE, SSX-1, -2, -3, -4, -5, -6, -7, -8, -9, HOM-TES-14/SCP-1, HOM-TES-85 and PRAME. Still other examples of CT antigens and the cancers in which they are expressed include SSX-2, and -4 (Neuroblastoma), SSX-2 (HOM-MEL-40), MAGE, GAGE, BAGE and PRAME (Malignant melanoma), HOM-TES-14/SCP-1 (Meningioma), SSX-4 (Oligodendrioglioma), HOM-TES-14/SCP-1, MAGE-3 and SSX-4 (Astrocytoma), SSX member (Head and neck cancer, ovarian cancer, lymphoid tumors, colorectal cancer and breast cancer), RAGE-1, -2, -4, GAGE-1-2, -3, -4, -5, -6, -7 and -8 (Head and neck squamous cell carcinoma (HNSCC)), HOM-TES14/SCP-1, PRAME, SSX-1 and CT-7 (Non-Hodgkin's lymphoma), and PRAME (Acute lymphoblastic leukemia (ALL), acute myelogenous leukemia (AML) and chronic lymphocytic leukemia (CLL)).

Other cancer biomarkers are not specific to a particular tissue or cell lineage. These include members of the carcinoembryonic antigen (CEA) family: CD66a, CD66b, CD66c, CD66d and CD66e. These antigens can be expressed in many different malignant tumors.

Still other cancer biomarkers are viral proteins and these include Human papilloma virus protein, and EBV-encoded nuclear antigen (EBNA)-1.

In some embodiments, the present invention provides methods of screening for a test compound that alters (e.g., increases or decreases) the presence of biomarkers. In some embodiments, test and/or candidate compounds are antisense agents (e.g., siRNAs, oligonucleotides, etc.) directed against biomarkers. In other embodiments, candidate compounds are antibodies that specifically bind to a biomarker.

In one screening method, candidate compounds are evaluated for their ability to alter biomarker presence, activity and/or expression by administering the compound to a subject harboring a tumor (e.g., an isogenic zebrafish harboring a transplanted tumor) and then assaying for the effect of the candidate compound on the presence or expression of the biomarker. In some embodiments, the effect of candidate compounds on expression or presence of a biomarker is assayed for by detecting the level of biomarker present in cells. In other embodiments, the effect of candidate compounds on expression or presence of a biomarker is assayed for by detecting the level of biomarker present in the extracellular matrix.

In other embodiments, the effect of candidate compounds on expression or presence of biomarkers is assayed by measuring the level of polypeptide encoded by the biomarkers. The level of polypeptide expressed can be measured using any suitable method, including but not limited to, those disclosed herein (e.g., antibodies, Western blotting, immunofluorescence, etc.

Specifically, in some embodiments, the present invention provides screening methods for identifying modulators, i.e., candidate or test compounds or agents (e.g., proteins, peptides, peptidomimetics, peptoids, small molecules or other drugs) that bind to proteins that generate biomarkers, have an inhibitory (or stimulatory) effect on, for example, biomarker expression and/or biomarker activity, or have a stimulatory or inhibitory effect on, for example, the expression or activity of a biomarker substrate. Compounds thus identified can be used to modulate the activity of target gene products either directly or indirectly in a therapeutic protocol, to elaborate the biological function of the target gene product, or to identify compounds that disrupt normal target gene interactions. Compounds that inhibit or enhance the activity, expression or presence of biomarkers find use in the treatment of cancers and/or tumors.

In one embodiment, the invention provides assays for screening candidate or test compounds that are substrates of a biomarker. In another embodiment, the invention provides assays for screening candidate or test compounds that bind to or modulate the activity of a biomarker.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; See, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (See, e.g., Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (See, e.g., Houghten, Biotechniques 13:412-421 (1992)), or on beads (See, e.g., Lam, Nature 354:82-84 (1991)), chips (See, e.g., Fodor, Nature 364:555-556 (1993)), bacteria or spores (See, e.g., U.S. Pat. No. 5,223,409; herein incorporated by reference), plasmids (See, e.g., Cull et al., Proc. Nad. Acad. Sci. USA 89:18651869 (1992)) or on phage (See, e.g., Scott and Smith, Science 249:386-390 (1990); Devlin Science 249:404-406 (1990); Cwirla et al., Proc. NatI. Acad. Sci. 87:6378-6382 (1990); Felici, J. Mol. Biol. 222:301 (1991)).

In one embodiment, an assay is a cell-based assay in which a cell (e.g., a transplanted tumor cell) that contains or is capable of generating a biomarker is contacted with a test compound, and the ability of the test compound to modulate the biomarker's expression or activity is determined. Determining the ability of the test compound to modulate biomarker expression or activity can be accomplished by monitoring, for example, changes in enzymatic activity or downstream products.

The ability of the test compound to modulate biomarker binding to a compound, e.g., a biomarker substrate, can also be evaluated. This can be accomplished, for example, by coupling the compound, e.g., the substrate, with a radioisotope or enzymatic label such that binding of the compound, e.g., the substrate, to a biomarker can be determined by detecting the labeled compound, e.g., substrate, in a complex.

Alternatively, the biomarker can be coupled with a radioisotope or enzymatic label to monitor the ability of a test compound to modulate biomarker binding to a substrate in a complex. For example, compounds (e.g., substrates) can be labeled with ¹²⁵I, ³⁵S¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which a biomarker is contacted with a test compound and the ability of the test compound to bind to the biomarker is evaluated.

Cell-free assays involve preparing a reaction mixture of the target gene protein and the test compound under conditions and for a time sufficient to allow the two components to interact and bind, thus forming a complex that can be removed and/or detected.

The interaction between two molecules (e.g., a biomarker and a compound) can also be detected, e.g., using fluorescence energy transfer (FRET) (See, e.g., Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In another embodiment, determining the ability of a biomarker to bind to a target molecule can be accomplished using real-time Biomolecular Interaction Analysis (BIA) (See, e.g., Sjolander and Urbaniczky, Anal. Chem. 63:2338-2345 (1991) and Szabo et al. Curr. Opin. Struct. Biol. 5:699-705 (1995)). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

To the extent that biomarkers can, in vivo, interact with one or more cellular or extracellular macromolecules, such as proteins, inhibitors of such an interaction are useful. A homogeneous assay can be used to identify inhibitors.

For example, a preformed complex of the target gene product and the interactive cellular or extracellular binding partner product is prepared such that either the target gene products or their binding partners are labeled, but the signal generated by the label is quenched due to complex formation (See, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, biomarkers can be used as a “bait” in a two-hybrid assay or three-hybrid assay (see, e.g., U.S. Pat. No. 5,283,317; Zervos et al., Cell 72:223-232 (1993); Madura et al., J. Biol. Chem. 268.12046-12054 (1993); Bartel et al., Biotechniques 14:920-924 (1993); Iwabuchi et al., Oncogene 8:1693-1696 (1993); and Brent WO 94/10300; each of which is herein incorporated by reference), to identify proteins that bind to or interact with biomarkers (“biomarker-binding proteins” or “biomarker-bp”). Such biomarker-bps can be activators or inhibitors of signals by the biomarkers or targets as, for example, downstream elements of a biomarker-mediated signaling pathway.

This invention further pertains to novel agents identified by the above-described screening assays. Accordingly, it is within the scope of this invention to further use an agent identified as described herein (e.g., a biomarker modulating agent, an antisense biomarker nucleic acid molecule, a siRNA molecule, a biomarker specific antibody, or a biomarker-binding substrate) in an appropriate model (e.g., isogenic zebrafish model described herein) to determine the efficacy, toxicity, side effects, or mechanism of action, of treatment with such an agent. Furthermore, novel agents identified by the above-described screening assays can be, e.g., used for treatments as described herein.

Thus, in its several aspects, the present invention provides methods for screening for anti-cancer agents; for the testing of anti-cancer therapies; for the development of drugs targeting pathways (e.g., associated with cancer); for the identification of new anti-cancer therapeutic targets (e.g., cancer biomarkers); the identification and diagnosis of cancerous (e.g., malignant) cells in pathology specimens; for the testing and assaying of cancer (e.g., a leukemia, lymphoma or solid tumor) drug sensitivity; and for the measurement of specific factors that predict drug sensitivity. The present invention can be used as a model to diagnose or test the sensitivity of a cancer (e.g., a leukemia, lymphoma or solid tumor) to known therapies; as a model for identification of new therapeutic targets for cancer treatment; and as a system to establish a tumor bank (e.g., comprising one or more of the tumor cell lines of Table 1) for testing new therapeutic agents for treatment of cancer. Also, the present invention provides compositions and methods that can be used in combination with existing compositions and methods (e.g., cancer databases (e.g., genomic databases of solid tumors (e.g., breast cancer tumors) or leukemias or lymphomas)), for improved discovery of anti-cancer agents.

The present invention is not limited by the type of cancer cell transplanted and/or characterized (e.g., with regard to response to test compound administered).

Examples of cancers that can be characterized (e.g., monitored for response to a test compound in an assay of the present invention) include, but are not limited to, lymphomas (e.g., Hodgkin's disease and non-Hodgkin's disease), leukemias (e.g., acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, and chronic lymphocytic leukemia), and sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The invention is also applicable to sarcomas and epithelial cancers, such as ovarian cancers and breast cancers, as well as to all solid tumors.

In some embodiments, the present invention provides characterization of the in vivo proliferation of transplanted cancer cell lines described herein (e.g., described in table 1). The in vivo proliferation of transplanted cancer cell lines can be accomplished by injection of transplanted cancer cell lines into subjects (e.g., genetically identical fish (e.g., isogenic zebrafish described herein)). Subjects can be injected with a varying number of cells and observed for cancer (e.g., tumor) formation (See, e.g., Examples 2-5). The injection can be by any method known in the art.

In some embodiments, subjects can be injected with cancer cell lines and observed for cancer formation (e.g., leukemia, lymphoma or solid tumors (See, e.g., U.S. Pat. App. No. 20040037815, herein incorporated by reference in its entirety for all purposes). Any cancers (e.g., tumors or myoproliferative disease) that form can be analyzed. Tests can be repeated (e.g., about 3, 5, 7, 10 or more times) to confirm the results. The phenotypes of the tumorigenic cells can thus be determined.

Other general techniques for formulation and injection of cells may be found in Remington's Pharmaceutical Sciences, 20th ed. (Mack Publishing Co., Easton, Pa.), hereby incorporated by reference in its entirety. Suitable routes may include parenteral delivery, including intramuscular, subcutaneous, intramedullary injections, as well as intrathecal, direct intraventricular, intravenous, intraperitoneal, or intraocular injections, just to name a few. For injection, the agents of the invention may be formulated in aqueous solutions, preferably in physiologically compatible buffers such as Hanks's solution, Ringer's solution, or physiological saline buffer.

Cancer cell lines (e.g., tumor cell lines of Table 1, or genetically modified versions thereof) can be subjected to tissue culture protocols known in the art (See, e.g., U.S. Pat. Nos. 5,750,376 and 5,851,832, Spector et al., Cells: A Laboratory Manual (Cold Spring Harbor Press, Cold Spring Harbor, N.Y., 1998)). Cancer cell lines can be genetically modified in vitro (e.g., in culture) to promote differentiation, cell death, or immunogenicity. For example, cancer cell lines can be modified to enhance expression of products that direct an immune response against the cancer. Alternatively, the cancer cell lines can be subjected to various proliferation protocols in vitro prior to genetic modification. The protocol used may depend upon the type of genetically modified caner cell lines or cancer cell line progeny desired. Once the cells have been subjected to the differentiation protocol, they can be assayed for expression of the desired protein.

Cancer cell line and cancer cell line progeny cultured in vitro or in vivo can be used for screening and identifying test compounds (e.g., small molecule inhibitors, pharmaceuticals, etc.) that can be used in or as an anticancer and/or antitumor therapeutic The ability to assay test compounds in vivo provided by the present invention provides the ability to monitor the effect of test compounds on both normal tissue and/or cells and cancer cell line population within a subject. For example, after introduction of cancer cells to a recipient subject, cancer cell survival, ability to form tumors, and biochemical and immunological characteristics can be examined.

Test compounds can be applied to cancer cells (e.g., in vivo or in vitro) at varying dosages, and the response of these cells monitored (e.g., over various time periods). Physical characteristics of these cells can be analyzed by observing cells by microscopy. The induction of expression of new or increased levels of proteins such as enzymes, receptors and other cell surface molecules can be analyzed with any technique known in the art. The techniques and methods described above for detection of cancer cell biomarkers find use in detecting gene and protein expression induced by test compound treatment.

Cancer cells of the present invention can be used to determine the effect of test compounds (e.g., small molecule inhibitors, pharmaceuticals, biological agents, etc.).

The ability of test compounds to alter (e.g., increase or decrease) cancer cell growth or maintenance, as well as the effect on normal cells and/or tissues, can be assayed. For example, in some embodiments, test compounds (e.g., from a library of compounds) are screened for their ability to alter (e.g., eliminate or inhibit growth of) cancer cells, while concurrently monitoring the effect on normal cells and/or tissue. Screening in this way permits the identification of compounds that can be utilized (e.g., independently, in a pharmaceutical composition, or co-administered) for treating cancer (e.g., inhibiting or eliminating cancer cells while having no harmful effect on normal cells and/or tissues).

In some embodiments, test compounds can be solubilized and added to cancer cells (e.g., in vitro (e.g., in the culture medium), or, in vivo (e.g., to a recipient subject that has received a cancer cell (e.g., tumor) graft)). In some embodiments, various concentrations of the test compound are utilized to determine an efficacious dose. In some embodiments, administration of the test compound is consistent over a period of time (e.g., administered to a recipient one, two or more times a day) so as to keep the concentration of the test compound constant.

Test compounds can be administered in vitro or in vivo at a variety of concentrations. For example, in some embodiments, test compounds are added to culture medium or to a subject so as to achieve a concentration from about 10 pg/ml to 10 mg/ml, or from about 1 ng/ml (or 1 ng/cc of blood) to 100 ng/ml (or 100 ng/cc of blood), although higher (e.g., greater than 10 mg/ml) and lower (e.g., less than 10 pg/ml) concentrations may also be used.

The effects of a test compound can also be identified on the basis of a significant difference relative to a control regarding criteria such as the ratios of expressed phenotypes, cell viability, proliferation rate, number of cancer cells, cancer cell activity upon transplantation in vivo, cancer cell activity upon transplantation in culture, cell cycle distribution of cancer cells, and alterations in gene expression.

It is contemplated that pharmaceutical compositions comprising a successfully identified test compound (e.g., a test compound identified that is capable of altering (e.g., inhibiting growth or eliminating) cancer cells (e.g., while concurrently not harming normal cells and/or tissues), analogue or mimetic can be administered systemically or locally to alter cancer stem cell growth and induce cancer (e.g., tumor) cell death in cancer patients.

Where combinations are contemplated, it is not intended that the present invention be limited by the particular nature of the combination. The present invention contemplates combinations as simple mixtures as well as chemical hybrids. An example of the latter is where a peptide or drug is covalently linked to a targeting carrier or to an active pharmaceutical. Covalent binding can be accomplished by any one of many commercially available crosslinking compounds.

It is not intended that the present invention be limited by the particular nature of the therapeutic preparation. For example, such compositions can be provided together with physiologically tolerable liquid, gel or solid carriers, diluents, adjuvants and excipients.

Once identified (e.g., using the compositions and methods of the present invention), a therapeutic preparation (e.g., comprising a test compound) can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

Such compositions are typically prepared as liquid solutions or suspensions, or in solid forms. Oral formulations for cancer usually will include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%.

The compositions are also prepared as injectables, either as liquid solutions or suspensions; solid forms suitable for solution in, or suspension in, liquid prior to injection may also be prepared.

The compositions of the present invention are often mixed with diluents or excipients which are physiological tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

It may be desirable to administer an analogue of a successfully identified test compound (e.g., a test compound identified that is capable of altering (e.g., inhibiting growth or eliminating) cancer cells while concurrently not harming normal cells and/or tissues). A variety of designs for such mimetics are possible. For example, cyclic peptides, in which the necessary conformation for binding is stabilized by nonpeptides, are specifically contemplated. (See, e.g., U.S. Pat. No. 5,192,746 to Lobl et al., U.S. Pat. No. 5,169,862 to Burke, Jr. et al., U.S. Pat. No. 5,539,085 to Bischoff et al., U.S. Pat. No. 5,576,423 to Aversa et al., U.S. Pat. No. 5,051,448 to Shashoua, and U.S. Pat. No. 5,559,103 to Gaeta et al., all hereby incorporated by reference, describe multiple methods for creating such compounds).

Synthesis of nonpeptide compounds that mimic peptide sequences is also known in the art. For example, Eldred et al., J. Med. Chem. 37:3882 (1994), describe nonpeptide antagonists that mimic the Arg-Gly-Asp sequence. Likewise, Ku et al., J. Med. Chem. 38:9 (1995) give further elucidation of the synthesis of a series of such compounds. Such nonpeptide compounds are specifically contemplated by the present invention.

The present invention also contemplates synthetic mimicking compounds that are multimeric compounds that repeat the relevant peptide sequence. As is known in the art, peptides can be synthesized by linking an amino group to a carboxyl group that has been activated by reaction with a coupling agent, such as dicyclohexyl-carbodiimide (DCC). The attack of a free amino group on the activated carboxyl leads to the formation of a peptide bond and the release of dicyclohexylurea. It may be important to protect potentially reactive groups other than the amino and carboxyl groups intended to react (e.g., the x-amino group of the component containing the activated carboxyl group can be blocked with a tertbutyloxycarbonyl group). This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble, matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

The methods of the present invention can be practiced in vitro, ex vivo, or in vivo.

### EXPERIMENTAL

The following examples are provided in order to demonstrate and further illustrate certain preferred embodiments and aspects of the present invention and are not to be construed as limiting the scope thereof.

### Example 1

### Materials and Methods

Animals. Zebrafish of the line brass were purchased in a local pet shop. The wild-type (AB) zebrafish were obtained from Dr. E. Weinberg (University of Pennsylvania, Philadelphia, Pa.) and maintained in the laboratory for >8 years. The golden strain of zebrafish was obtained from Dr. H. G. Frohnhoefer (MPI for Developmental Biology, Germany). During the entire study, fish were maintained in 20-L acrylic tanks connected to a closed water recirculation system with 50 to 60 fish in each. Fish were maintained in standard conditions (See, e.g., Westerfield, The Zebrafish Book. Guide for the laboratory use of zebrafish (Danio rerio). 3rd ed. Eugene (OR): University of Oregon Press; 1995). Briefly: 14/10-hour light/dark cycle, T=26±1° C. Tetramin (Tetra, Melle, Germany) and Ovo-vit (QXL, Poznan, Poland) were used as a basal diet supplemented with nauplii Artemia salina.

Generation of clonal homozygous zebrafish. Clones of homozygous zebrafish CB1 and CW1 derived from brass and AB strains of zebrafish, respectively, were obtained by heat shock procedure as described previously (See, e.g., Streisinger et al., Nature (1981) 291:293-6) with some modifications. Briefly, eggs from female AB and brass strains of zebrafish were fertilized by UV-inactivated sperm from male zebrafish of golden and AB strains, respectively. After 13-minute incubation at 28.5° C. eggs underwent a 2-minute heat shock at 41.4° C. to block the first cleavage (the present invention is not limited to heat shock, indeed, other methods can also be used to shock the eggs (e.g., prior to a first cleavage) including, but not limited to, cold-shock and or hyrdrostatic pressure shock). This procedure led to the development of a homozygous diploid fish. Eggs obtained from a raised to maturity homozygous female fish of either AB or brass strain underwent the second round of UV-sperm fertilization/heat shock procedure as described above. The offspring obtained from each homozygous female were genetic copies of each other (i.e., clones of homozygous fish). The further maintenance of homozygous strains was carried out by crossing fish within each clone. Mating of the fish that belonged to different homozygous clones led to the generation of an isogeneic fish strain consisting of genetically identical but not homozygous fish.

Tumor induction. Sixty-five 2.5-month-old fish from the clonal CB1 line were used for tumor induction by continuous immersion in N-nitrosodiethylamine (DEN) solution (100 ppm) during the 8 weeks. During DEN, exposure fish were maintained in 20-L acrylic tanks equipped with a mechanical filter and an automatic heater. The filters were cleaned twice weekly. The complete exchange of water containing the carcinogen was done every 2 weeks. The water temperature was maintained at 26±0.5° C. Following completion of DEN exposure the fish were transferred to a 20-L system tank and remained under observation for up to 9 months.

Tumor transplantation. The tumor tissue for further grafting was obtained from fish with grossly visible heoplasms located in the abdomen. These fish were euthanized by 0.015% tricaine (Sigma, Munich, Germany) solution and were opened ventrally with iridectomy scissors. Tumors were gently separated from surrounding tissues and placed into cold (4° C.) Ringer solution with antibiotics (100 units/mL penicillin and 0.1 mg/mL streptomycin) for 2 to 30 minutes (until transplantation). In the meantime, 2.5- to 5-month-old syngeneic recipients were placed in 0.015% tricaine solution in groups of three to six fish. Immediately after reaching immobility, the fish were transferred individually onto a wet sponge for further transplantation of tumor tissue. I.p. transplantation of tumor tissue was carried out by a small punch using a thick syringe needle (2 mm) in the left side of abdomen. A piece(s) of a tumor (1-2 mm³) in 10 to 20 μL of Ringer solution was injected into abdomen through this punch by a fire-polished Pasteur pipette. The wound caused by the punch was not stitched. In i.m. transplantation, pieces of tumor were injected into dorsal muscles in the area of the dorsal fin by a syringe needle equipped with a well-fitting metal plunger. After the transplantation, fish were transferred into dechlorinated tap water containing 10 μg/mL methylene blue for 24 hours followed by their return to a system tank. To transplant tumors to 7- to 14-day larvae, the tumors removed from the donors were mechanically minced in 200 to 300 μL of cold (4° C.) PBS and filtered through 40-μm mesh (Becton Dickinson Labware, Mountain View, Calif.). After a short centrifugation, the pellet consisting of single cells and cell clusters (5-20 cells) was resuspended in 25 μL PBS and kept on ice until transplantation. Using a flexible syringe needle “MICROFIL” (WPI, Inc., Tonasket, Wash.), about 5 μL cell suspension was placed into a glass micropipette with an external diameter at the tip of 30 μm without a filament and made with a vertical puller. Recipient larvae were placed into 0.015% tricaine solution until they became immobile. Then, the larvae were transferred onto a wet filter paper in groups of 25 animals. After i.p. injection of about 50 to 100 nL (depending on recipient size) of cell suspension containing 2.5 to 3×10⁶ cells per 10 μL, all the larvae were returned to clean tap water. The animals in the control group were injected with the same volume of Ringer solution.

Cryopreservation. Large tumors nodules that underwent 5 to 10 passages were extracted from fish abdomens and cut into 2 to 3 parts (˜5×5×5 mm). Each part was placed in a 0.5-mL cryopreservation tube (Sarstedt, Nuembrecht, Germany) containing 400 μL DMEM supplemented with 20% FCS and 10% DMSO (Sigma, Munich, Germany) and then was further cut with scissors into pieces of about 1×1 mm. The tubes were kept at 4° C. for 30 minutes followed by overnight storage at −80° C. and then transferred to liquid nitrogen for a long-term storage. Thawing was carried out by a quick transfer of the tubes from liquid nitrogen to a water bath at 30° C. to 32° C. followed by a replacement of cryopreservation medium with DMEM without FCS and DMSO. After thawing, samples were kept at 4° C. until transplantation as described above.

Histology. After 24 to 72 hours of fixation in 4% solution of neutral paraformaldehyde, all primary and transplanted tumors together with adjacent abdominal organs underwent standard histologic processing. Tissue slices (3-4 μm) were stained by H&E or, if necessary, with other staining methods. Photographs of the slides were obtained by Nikon Coolpix 4500 digital camera, mounted with an optical adapter on the microscope BIOLAM-I (LOMO, St. Petersburg, Russia).

Determination of mean survival time tumor-bearing fish. The mean survival time of tumor-bearing fish was assessed in the two most fast growing tumor lines zt23 and s1 implanted i.p. to syngeneic 3.5-month-old fish or to 12-day-old syngeneic larvae (zt23 only). Each experimental and control group consisted of either 10 adult females or 29 larvae. During the observation, each group of fish was kept in a separate 20-L system tanks (for adults) or in 400-mL beakers (for larvae) at 26° C. and fed a standard diet or nauplii Artemia, respectively. The assessment of survival time continued until the death of the last fish in each experimental group.

### Example 2

### Generation and Characterization of Transplantable Tumor Cell Lines

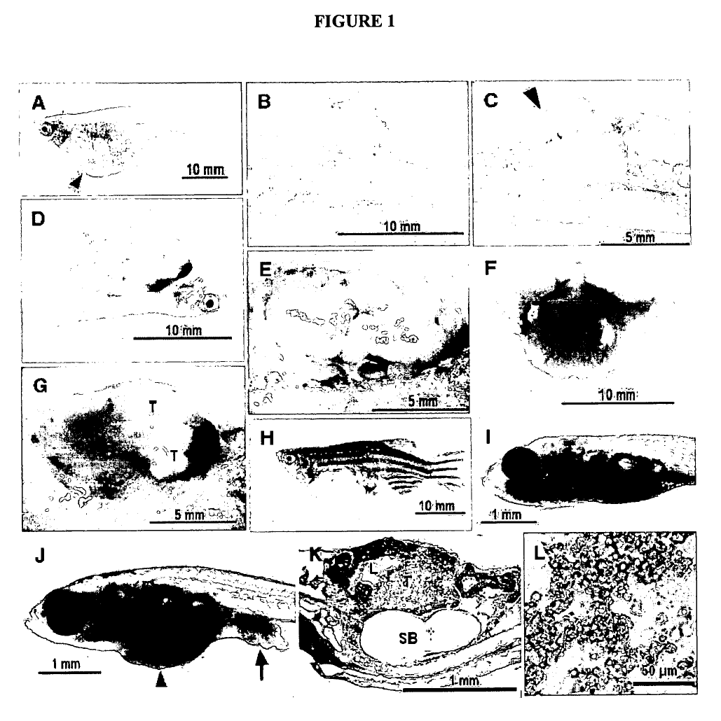

Treatment of zebrafish with DEN resulted in 35 primary tumors localized in the abdominal area and reaching from 3 to 10 mm in diameter. The grossly visible tumors began to emerge in fish 3 to 7 months after the beginning of exposure to DEN. Four spontaneous tumors diagnosed as acinar cell carcinomas of the pancreas were detected in 10.5- to 18-month-old female CB1 fish (See, e.g., FIG. 1G).

Of 29 transplanted DEN-induced and spontaneous tumors, 19 underwent from 3 to 25 consecutive passages in syngeneic fish. The other tumors were lost as a result of the recipient's death or because of a tumor growth arrest after several passages. In the majority of cases, i.p. or i.m. transplanted tumors showed almost synchronous growth in fish inoculated simultaneously. The efficacy of tumor transplantation to both sites was close to 100% as would be expected in syngeneic recipients. Features of the transplanted tumor lines generated and characterized during the development of the present invention are shown in Table 1, below.

TABLE 1
Features of transplantable tumor lines in zebrafish.

| | | | | Interval | Duration | | | | |
|---|---|---|---|---|---|---|---|---|---|
| | | | | between | of tumor | | Growth in | | |
| | Tumor | Tumor | No. | passages | maintenance | | isogenic | RTLA | Histologic diagnosis of |
| No. | line | origin | passages* | days^{†} | (mo)^{‡} | Invasiveness^{§} | fish | no.^{\|\|} | tumor lines |
| 1 | zt2 | DEN induced | 11 | 45-60 | 18.0 | +/− | Yes | 7606 | Hepatoblastoma and |
| | | | | | | | | | Hepatocellular Carcinoma |
| 2 | zt6 | DEN induced | 10 | 45-60 | 17.5 | +/− | Yes | 7607 | Hepatoblastoma and |
| | | | | | | | | | Hepatocellular Carcinoma |
| 3 | zt8 | DEN induced | 13 | 30-45 | 17.3 | +/− | Yes | 7609 | Hepatocellular Carcinoma |
| 4 | zt9 | DEN induced | 13 | 30-45 | 17.3 | +/− | Yes | 7610 | Hepatocellular Carcinoma |
| 5 | zt10 | DEN induced | 7 | 65-80 | 17.3 | +/− | Yes | 7611 | Cholangiocarcinoma |
| 6 | zt12 | DEN induced | 7 | 60-90 | 17.3 | +/− | Yes | 7612 | Poorly differentiated |
| | | | | | | | | | hepatocellular carcinoma |
| 7 | zt15 | DEN induced | 12 | 30-45 | 17.3 | +/− | Yes | 7613 | Hepatocellular carcinoma |
| 8 | zt16 | DEN induced | 8 | 50-75 | 17.0 | +/− | Yes | 7614 | Poorly differentiated |
| | | | | | | | | | hepatocellular carcinoma |
| 9 | zt18 | DEN induced | 13 | 40-50 | 16.7 | +/− | Yes | 7615 | Poorly differentiated |
| | | | | | | | | | hepatocellular carcinoma |
| 10 | zt20 | DEN induced | 8 | 45-75 | 16.5 | − | Yes | 7616 | “Minimal deviation” |
| | | | | | | | | | hepatoma |
| 11 | zt21 | DEN induced | 12 | 40-50 | 16.5 | +/− | Yes | 7617 | Hepatoblastoma |
| 12 | zt23 | DEN induced | 25 | 10-20 | 16.5 | + | Yes | 7618 | Pancreatic acinar cell |
| | | | | | | | | | carcinoma |
| 13 | zt28 | DEN induced | 11 | 45-60 | 16.3 | +/− | Yes | 7619 | Hepatocellular carcinoma |
| 14 | zt29 | DEN induced | 12 | 30-45 | 16.3 | +/− | Yes | 7620 | Hepatocellular carcinoma |
| | | | | | | | | | (with spindle cell pattern) |
| 15 | zt34 | DEN induced | 18 | 20-30 | 16.2 | +/− | Yes | 7621 | Poorly differentiated |
| | | | | | | | | | hepatocellular carcinoma |
| 16 | s1 | Spontaneous | 14 | 20-30 | 12.5 | + | Yes | 7605 | Pancreatic acinar cell |
| | | | | | | | | | carcinoma |
| 17 | s3 | Spontaneous | 5 | 50-75 | 10.0 | − | NT | | Pancreatic acinar cell |
| | | | | | | | | | carcinoma |
| 18 | s10 | Spontaneous | 3 | 30-45 | 2.0 | − | NT | | Pancreatic acinar cell |
| | | | | | | | | | carcinoma |
| 19 | s11 | Spontaneous | 3 | 30-45 | 1.5 | − | NT | | Pancreatic acinar cell |
| | | | | | | | | | carcinoma |

Abbreviation:
NT, not tested.
*Number of tumor passages up to date.
^{†}Average interval between two consecutive tumor transplantations (recipient's age ranged between 2.5 and 6 mos).
^{‡}Total duration of the tumor line maintenance beginning from the first passage.
^{§}(+), infiltrative growth; (+/−), invasive growth detected during histologic analysis only; (−), tumor invasion is not detected.
^{||}Identification no. at the Registry of Tumors in Lower Animals.

The majority of tumors were characterized by a moderate growth rate and required further passage approximately every 1 to 2 months. The others tumor lines remained either slow growing during the entire period of observation (e.g., lines zt10, zt12, zt16, zt20, and s3) or, by contrast, had a very high growth rate and required transplantation every 2 to 4 weeks (e.g., lines s1, zt23, and zt34). Invasiveness also varied among different transplanted tumor lines. For example, tumor lines zt23 and s1 appeared the most invasive. Macroscopically, these tumors were composed of an amorphous jelly-like mass of milky-white (e.g., s1) or yellowish (e.g., zt23) color, filling essentially the entire abdominal cavity (See, e.g., FIG. 1D).

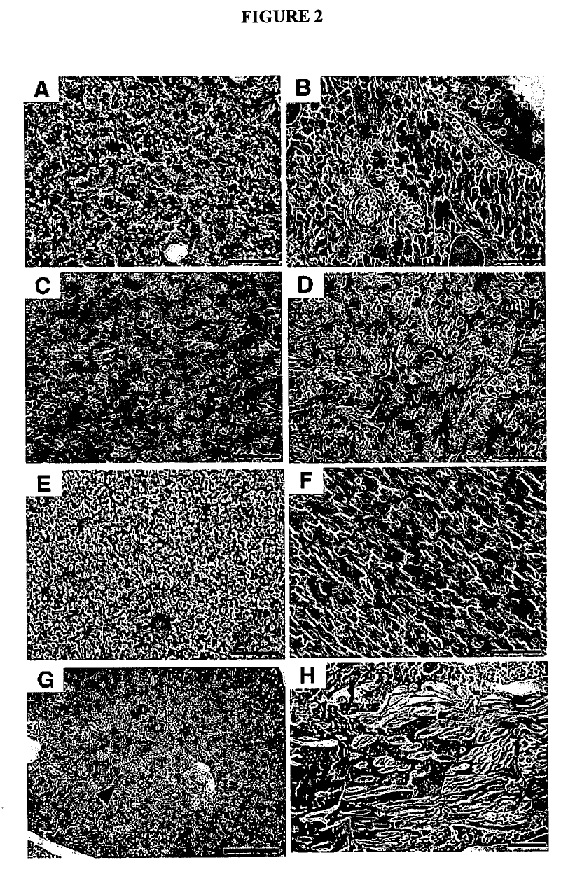

The liver, ovaries, and abdominal wall were the most common sites of invasion of these tumors. Less invasive tumors typically grew as solitary pink or yellowish solid smooth nodules up to 1 cm in diameter, distinctively separated from surrounding abdominal organs (See, e.g., FIG. 1E). These nodules in general were connected to the host vascular system by a blood vessel stalk. The majority of these tumors also showed invasive growth. Invasion into surrounding organs could be determined using microscopic assessment (See, e.g., FIG. 2H). Minimal deviation hepatoma (zt20) grew as a relatively benign noninvasive tumor, forming structures grossly similar to those of normal liver with regard to shape, color, and consistency (See, e.g., FIG. 1F).

### Example 3

### Transplantation of Tumor Cell Lines

During multiple serial transplantations, tumor lines retained a close similarity to the histologic structure of the primary tumors. Most of the DEN-induced tumor lines were diagnosed as a typical hepatocellular (e.g., lines zt8, zt9, zt12, zt15, zt16, zt18, zt28, zt29, and zt34; See, e.g., FIG. 2C) and cholangiocellular (zt10; See, e.g., FIG. 2D) carcinomas. The morphology of these types of tumors has been described in detail for various fish species, including zebrafish (See, e.g., Khudoley, J Natl Cancer Inst Monogr 1980; 65:65-70; Boorman et al., Toxicol Pathol 1997; 25:202-10). Hepatoblastomas (e.g., lines zt2, zt6, and zt21) were typically composed of undifferentiated embryonal-like cells, sometimes in combination with hepatocarcinoma cell areas that display an arrangement suggestive of a rosette-like formation. The histology of these neoplasms resembled tumors observed in Fundulus heteroclitus from a creosote-contaminated area (See, e.g., Vogelbein et al., Cancer Res 1990; 50:5978-86). The minimal deviation hepatoma zt20 was composed of essentially normal hepatocytes with round unimorfic nuclei and vacuolated cytoplasm (See, e.g., FIG. 2E). This tumor is thought to represent the zebrafish analogue of minimal hepatomas described in rats (See, e.g., Morris, Prog Exp Tumor Res 1963; 3:370-411). DEN-induced acinar cell carcinomas in pancreas (e.g., line zt23) grew as large fields comprising separate strongly basophilic small cells infiltrating the gaps between adjacent organs (See, e.g., FIG. 1K). In some cases, liver metastases were observed after i.p. grafting of this tumor.

### Example 4

### Histology of Transplanted Tumor Lines

Histology of liver metastases observed after grafting of tumor was similar to the tumor that they were derived from (See, e.g., FIG. 2G). Spontaneous acinar cell carcinomas in pancreas (e.g., line s1, s3, s10, and s11) grew as solid sheets formed by large polygonal cells with moderately polymorphic nuclei, with prominent nucleoli and basophilic cytoplasm that contained bright eosinophilic secretory granules (See, e.g., FIG. 2F). In some areas, the structure of these tumors resembled normal pancreatic tissue in zebrafish (See, e.g., FIG. 2B). Histologic diagnoses of all tumor lines were validated by the Registry of Tumors in Lower Animals (Sterling, Va.).

Signs of advanced stages of tumor growth, such as significant enlargement (e.g., often asymmetric) of the abdomen (See, e.g., FIG. 1A), development of ascit in the abdominal cavity, tumor penetration of the abdominal wall (See, e.g., FIG. 1C), cachexia, or various combinations of these symptoms, were used as criteria for expediency of the next i.p. tumor passage in adult fish. By contrast, slow growing (e.g., lines zt10, zt12, zt16, and s3) or relatively benign (e.g., zt20) tumor lines underwent consecutive passages approximately every 2 to 3 months without dependency upon macroscopic signs of tumor progression. Tumor size was the main criteria for the next tumor passage after i.m. transplantation (See, e.g., FIG. 1B).

### Example 5

### Tumor Growth and Lifespan of Tumor-Bearing Fish

Transplantation of several moderately and fast growing tumor lines (e.g., lines zt9, zt18, and zt23) to wild-type (AB) zebrafish failed. In contrast, transplantation of the majority of tumor lines to isogeneic zebrafish was successful (See, e.g., Table 1, above; and FIG. 1H). However, the growth rate of tumors transplanted to isogenic hosts was typically ˜50% slower than that in their syngeneic counterparts.

The frequency of successful transplantation of tumor lines zt23, zt34, and s1 recovered after cryopreservation was around 100%, 100%, and 33%, respectively. Tumors that developed after cryopreservation showed invasive growth and retained a histologic structure of the corresponding tumor lines.

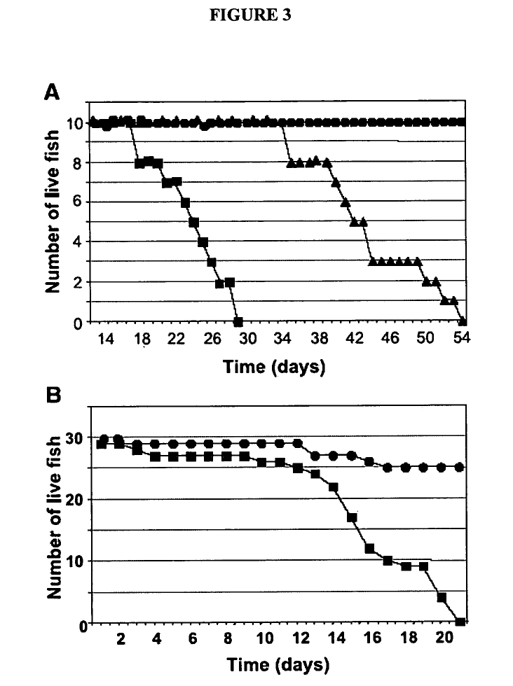

The mean survival time of adult fish after i.p. tumor grafts was 25±4.0 and 44.7±6.2 days for tumor lines zt23 and s1, respectively (See, e.g., FIG. 3A). No fish death occurred in a control group during the entire period of observation. The mean survival time of 12-day larvae i.p. injected with zt23 tumor cells was 15±4.2 days (See, e.g., FIG. 3B).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of identifying a test compound useful for treating cancer comprising:
a) providing a subject, wherein said subject is a cloned, isogenic zebrafish harboring transplanted tumor cells;
b) administering a test compound to said subject;
c) monitoring said subject; and
d) identifying a test compound that alters a characteristic of said subject.

2. The method of claim 1, wherein said transplanted tumor cells are selected from the group consisting of hepatobastoma, hepatocellular carcinoma, cholangiocarcinoma, minimal deviation hepatoma, and pancreatic acinar cell carcinoma.

3. The method of claim 1, wherein said characteristic is transplanted tumor cell growth.

4. The method of claim 1, wherein said characteristic is transplanted tumor cell mass.

5. The method of claim 1, wherein said characteristic is zebrafish mass.

6. The method of claim 1, wherein said characteristic is zebrafish mortality.

7. The method of claim 1, wherein said characteristic is expression of a cancer biomarker.

8. The method of claim 7, wherein said expression is selected from the group consisting of gene expression and protein expression.

9. The method of claim 1, wherein said characteristic is the number of transplanted tumor cells.

10. The method of claim 1, wherein microscopy is used for said monitoring.

11. The method of claim 10, wherein said microscopy is selected from the group consisting of direct microscopy, dissecting microscopy, fluorescent microscopy, and inverted microscopy.

12. The method of claim 1, wherein said zebrafish is selected from the group consisting of zebrafish embryos, zebrafish larvae and adult zebrafish.

13. The method of claim 1, wherein said zebrafish are present within a multi-well plate.

14. The method of claim 1, wherein monitoring comprises histological techniques.

15. The method of claim 14, wherein said histological techniques are selected from the group consisting of fixation of the material, decalcination and paraffination, and preparation and staining tissue sections.

16. A tumor cell line derived from an isogenic zebrafish clone, wherein said tumor cell line is selected from the group consisting of hepatobastoma, hepatocellular carcinoma, cholangiocarcinoma, minimal deviation hepatoma, and pancreatic acinar cell carcinoma.

17. The tumor cell line of claim 17, wherein said cell line retains the ability to form tumors in a subject after a cycle of freezing and thawing.

18. A method of generating an isogenic zebrafish comprising:
a) providing eggs from female strains of AB or brass strains of zebrafish fertilized by UV-inactivated sperm from male zebrafish,
b) heat-shocking said eggs, wherein said heat-shocking blocks the first cleavage of said fertilized eggs;
c) obtaining eggs from female homozygous diploid fish resulting from steps (a) and (b); and
d) repeating steps (a) and (b) with the eggs of step (c) to generate clones of homozygous fish; and
e) crossing two different homozygous clones of the same maternal origin, thereby generating an isogenic zebrafish.

19. An isogenic zebrafish generated according to the method of claim 18.

20. A clonal isogenic small teleost fish comprising heterologous cells and/or tissue.
